(19) United States
(12) Patent Application Publication
(10) Pub. No.: US 2009/0249566 A1
(43) Pub. Date: Oct. 8, 2009

(54) Disposable Container Top Cleaner

(76) Inventor: Jeremy Chalmers, Philadelphia, MS (US)

Correspondence Address: FRASCOGNA COURTNEY, PLLC, P.O.BOX 23126, JACKSON, MS, 39225-3126, US

(21) Appl. No.: 12/098,705

(22) Filed: Apr. 7, 2008

(51) Int. Cl. A47L 25/00

(52) U.S. Cl. 15/104.001

(57) ABSTRACT

A disposable container top cleaner is disclosed. This invention may be used to wipe and disinfect the lids of cans, bottles, and other containers prior to use in food preparation or consumption. The invention may be cheaply produced and disposed of after a limited number of uses.

### FIELD OF THE INVENTION

This invention relates generally to container top cleaners and specifically to container top cleaners that can be disposed of after a limited number of uses.

### BACKGROUND OF THE INVENTION

The beverage industry produces millions of gallons of sodas, juices, and alcoholic beverages a year. Many of these drinks are stored in disposable cans or bottles and shipped to consumers. Often, a consumer drinks the beverage by placing his mouth on the lid of the can or mouth of the bottle. This can be undesirable because the beverage container may be exposed to chemicals, pests, and germs during the long trip from the factory to the end consumer. Furthermore, many consumers wish to drink directly from the container for convenience, but are concerned to do so because of an inability to determine what kind of potentially unpleasant substances may be present on the container.

Several container lid cleaners have been created in an attempt to solve this problem, but all have some design deficiencies. Large automated cleaners help clean containers at the factory, but are unavailable to the end consumer. Hand held container top cleaners are known in the art. However, most must be used in combination with a disposable disinfectant cloth and require the user to carry them at all times in case a drink is needed, which is impractical. Still other lid cleaners are designed to be included as part of the container; however, most containers are not produced with these cleaners attached. Therefore, these cleaners are practically unavailable to the average consumer.

Drinks are often purchased by consumers with little or no forethought and as a matter of impulse. For this reason, the lid cleaners must be available to the impulse consumer for effective marketing. Because this is the case, lid cleaners must be placed conspicuously in stores so that consumers will think to purchase them. The lid cleaners currently available are too heavy to be easily stored in the proper store locations, such as attached by suction cups to the glass of drink cases or to the front of vending machines. Also, these lid cleaners are shaped in a manner that makes them unsuitable for use on the mouth of a bottle.

Therefore, what is needed is a disposable container top cleaner which can be cheaply produced, is usable on many types of containers, is disposable, and is very small. Furthermore, other desirable features and characteristics of the present invention will become apparent when this background of the invention is read in conjunction with the subsequent detailed description of the invention, appended claims, and the accompanying drawings.

### SUMMARY OF THE INVENTION

The present invention advantageously fills the aforementioned deficiencies by providing a disposable beverage container top cleaner which can be used to clean multiple types of container tops, can be cheaply and easily produced, is disposable, and is small enough to be easily displayed on or near drink dispensers to attract the attention of impulse consumers.

In one particular embodiment of the present invention, a rigid square of material is attached to a flexible rectangle of material. The user may then turn the rigid portion of the device into a position perpendicular to the top of a can and parallel to the lip of said can. The user may then place the rigid portion against the inside lip of the container, bend part of the flexible portion over the lip of the container, place part of the flexible portion against the top of the container, pinch the flexible portion against the outside lip of the container, wipe both the top and outside surfaces of the container lip clean of undesirable matter, and discard the device.

In another embodiment, the flexible material is treated with a disinfectant. When the user wipes the top and outside surfaces of the container, any germs on those surfaces will be destroyed by the disinfectant. The user may then discard the device.

In still another embodiment, the shape of the rigid material is curved or otherwise altered to fit the contour of the lip of a specific type of can or the mouth of a bottle. The user may then easily disinfect either surface and discard the device.

The present invention now will be described more fully hereinafter with reference to the accompanying drawings, which are intended to be read in conjunction with both this summary, the detailed description, and any preferred and/or particular embodiments specifically discussed. This invention may, however, be embodied in many different forms and should not be construed as limited to the embodiments set forth herein; rather, these embodiments are provided by way of illustration only and so that this disclosure will be thorough, complete and will fully convey the full scope of the invention to those skilled in the art.

### BRIEF DESCRIPTION OF THE DRAWINGS

The drawings contained herein exemplify one of the embodiments of the claimed invention. It should be noted that the invention is not limited to the embodiment shown. The embodiment shown is purely an example, and the invention is capable of many variations of said embodiment. In the drawings,

FIG. 1 is an exploded view of an embodiment of a disposable beverage container top cleaner.

FIG. 2 is a planar view of an embodiment of a disposable beverage container top cleaner.

FIG. 3 illustrates a user pinching an embodiment of a disposable container top cleaner against the inside lip, outside lip, and top of a beverage container prior to wiping.

It should be noted that due to the many and varied embodiments of the present invention that can be created by attaching the components in slightly different positions relative to each other, the figures listed above are not drawn to a specific scale.

### DETAILED DESCRIPTION OF THE INVENTION

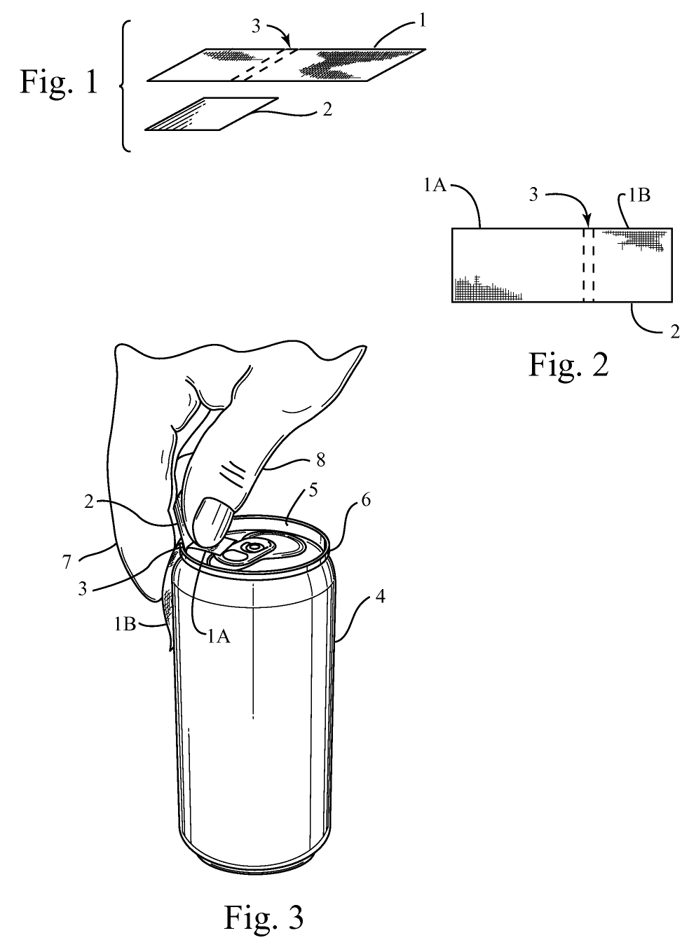

FIG. 1 illustrates an exploded view of an embodiment of the disposable container top cleaner. The disposable container top cleaner comprises a cleaning element 1, a structure element 2, and an adhesive area 3.

The cleaning element 1 is depicted as a rectangle for the purpose of simplicity; however, many different shapes may be used. The cleaning element 1 is used to clean the upper portion of a container. It also provides a place for the user to grip the device for greater control during use. In the preferred embodiment, the cleaning element 1 should be made from a flexible material that may be cheaply manufactured such as paper, vinyl, plastic, cloth, or similar materials. Additionally, in the preferred embodiment, the cleaning element 1 is treated with cleansing chemicals. Cleansing chemicals are any chemicals that may be used to kill bacteria, viruses, or assist in the removal of unwanted matter from a surface, and cause no appreciable harm to humans when consumed orally in trace amounts.

In a separate embodiment, the cleansing chemicals on the cleaning element 1 may be omitted from the disposable container top cleaner. This has the advantage of further decreasing the production costs of the invention. The disadvantage of omitting the cleansing chemicals is the loss of ability to disinfect the surfaces of a container. In this embodiment, the disposable container top cleaner may only be used to remove visible matter from the container surface and will have little effect on viruses or bacteria.

In the preferred embodiment, the structure element 2 should be made of a cheaply produced rigid material such as cardboard, plastic, aluminum, wood, or similar materials. The structure element 2 is depicted as a square for simplicity, however, many different shapes may be used including shapes designed to fit the contours of a specific type of container lid.

A portion of the structure element 2 is attached to a portion of the cleaning element 1 using a low cost durable adhesive. These portions are referred to as the connection portions. The connection portions are connected in the adhesive area 3. The adhesive area 3 contains a band of adhesive between the dotted lines in FIG. 1. The adhesive may be placed on the upper side of the structure element 2, on the lower side of the cleaning element 1, or both prior to assembly of the device. The distance between the dotted lines should be based on the height of the lip of the container to be cleaned with the particular embodiment of the device. The cleaning element 1 should not be connected to the structure element 2 on either side of the adhesive area 3 for reasons discussed below. The position of the adhesive area may be adjusted to allow the device to better fit the container to be cleaned.

FIG. 2 illustrates a planar view of the preferred embodiment of the disposable container top cleaner. The cleaning element 1 and structure element 2 are pressed together and attached by an adhesive present in the adhesive area 3. The cleaning element is subdivided into the top cleaning portion 1A and the side cleaning portion 1B.

FIG. 3 illustrates a method of applying the disposable container top cleaner to a container 4 with an inside lip 5, and outside lip 6, and a top. In this method, the user turns the structure element 2 into a position perpendicular with the top of the container. The unattached top cleaning portion 1A should extend below the structure element 2, and the unattached side cleaning portion 1B should extend above the structure element 2.

The top cleaning portion 1A of the cleaning element 1 should then be bent in a way that allows the top cleaning portion 1A to be placed in contact with the top surface of the container. The connection portion of the cleaning element 1 should be placed between the inside lip 5 of the container 4 and the connection portion of the structure element 2. The side cleaning portion 1B of the cleaning element 1 should then be bent over the lip of the container 4 and placed in contact with the outside lip 6 of the container 4.

The user's thumb 8 then presses the disposable container top cleaner against the inside lip 5 of the container 4. The thumb 8 also presses the top cleaning portion 1A of the cleaning element against the top of the container. The user's forefinger 7 is then placed against the side cleaning portion 1B of the cleaning element 1 resting on the outside lip 6 of the container 4. The user may then use the thumb 8 and forefinger 7 to compress the cleaning element 1 against the inside lip 5 and outside lip 6 of the container 4. The user may also apply downward force to the top cleaning portion 1A of the cleaning element 1 to press the cleaning element 1 against the top of the container. The user may then move the disposable container top cleaner around the top of the container 4 while maintaining contact with the lip of the container. This has the effect of wiping and disinfecting both the top, inside lip 5, and outside lip 6 of the container 4. In an alternate embodiment, the user may reverse his grip by placing his forefinger 7 against the structure element 2 and the top cleaning portion 1A of the cleaning element 1 and thumb 8 against the side cleaning portion 1B of the cleaning element 1 prior to wiping. The user may then drink from the cleaned container and dispose of the disposable container top cleaner.

The present invention should not be construed as being limited to cleaning beverage cans. Any container may be cleaned with the present invention including but not limited to soup cans of various sizes, beverage bottles, and any other container used to store food or drink. Additionally, the present invention may be used to clean the lips of containers used to store non-edible items. By way of example, the present invention could be used to remove excess liquid from any reusable container including, but not limited to, soap dispensers, oil cans, and chemical sprayers. This would prevent excess liquid from remaining on the container lip after the liquid has been dispensed.

The present invention should not be limited by the relative lengths or placement of the structure element 2 or cleaning element 1, nor should the present invention be considered limited to the apparent scale as indicated in the accompanying drawings. Different placements of the structure element 2 will affect the length of the top cleaning portion 1A and the side cleaning portion 1B of the cleaning element 1. The length of the cleaning element 1 may be reduced or elongated and the position of the structure unit 2 may be adjusted to better fit the contours and other elements specific to the container to be cleaned.

While the present invention has been described above in terms of specific embodiments, it is to be understood that the invention is not limited to these disclosed embodiments. Many modifications and other embodiments of the invention will come to mind of those skilled in the art to which this invention pertains, and which are intended to be and are covered by both this disclosure and the appended claims. It is indeed intended that the scope of the invention should be determined by proper interpretation and construction of the appended claims and their legal equivalents, as understood by those of skill in the art relying upon the disclosure in this specification and the attached drawings.

I claim:

1. A disposable container top cleaner for cleaning a plurality of surfaces of the upper portion of
a container comprising;
an structure element made of a rigid material,
further comprising a connection portion; and
a cleaning element made of a flexible material,
further comprising a top cleaning portion and a side cleaning portion,
for cleaning the upper portion of a container, and
said cleaning element further comprising a connection portion;
wherein the connection portion of the structure element is attached to the connection portion of the cleaning element.

2. The disposable container top cleaner of claim 1,
further comprising an adhesive,
wherein the adhesive is interposed between the connection portion of the cleaning element and the connection portion of the structure element.

3. The disposable container top cleaner of claim 1, wherein; the structure element is comprised of cardboard.

4. The disposable container top cleaner of claim 1, wherein; the cleaning element is comprised of paper.

5. The disposable container top cleaner of claim 1, wherein; the cleaning element is comprised of cloth.

6. The disposable container top cleaner of claim 1, wherein; the cleaning element further comprises cleansing chemicals.

7. The disposable container top cleaner of claim 1, wherein; the structure element's shape is curved to conform to the shape of a container surface.

8. A method of cleaning a plurality of surfaces of the upper portion of a container with a
disposable container top cleaner comprising;
manipulating a disposable container top cleaner,
said disposable container top cleaner comprising a structure element comprising a connection portion, and a cleaning element comprising a connection portion, a top cleaning portion, and a side cleaning portion; and
wherein the connection portion of the structure element is attached to the connection portion of the cleaning element;
said manipulation further comprising;
moving the connection portion of the structure element into a position perpendicular to the upper portion of a container further comprising a top, inside lip, and outside lip,
placing the top cleaning portion of the cleaning element in contact with the top of the container, placing the connection portion of the cleaning element in contact with the inside lip of the container,
placing an side cleaning portion of the cleaning element against the outside lip of the container;
pressing the cleaning element against the top, inside lip, and outside lip of the container by applying force to the structure element; and
wiping the cleaning element of the disposable container top cleaner against the top, inside lip, and outside lip of the container.

9. The method of cleaning a plurality of surfaces of the upper portion of a container of claim 8, further comprising; pressing the cleaning element of the disposable container top cleaner against the container surfaces by applying force to said cleaning element.

10. The method of cleaning a plurality of surfaces of the upper portion of a container of claim 8, wherein, the cleaning element further comprises cleansing chemicals.
